(19) United States
(12) Patent Application Publication
(10) Pub. No.: US 2019/0069783 A1
(43) Pub. Date: Mar. 7, 2019

(54) AUTOMATIC NORMALIZATION OF INTRAVASCULAR PRESSURE DEVICES

(71) Applicant: KONINKLIJKE PHILIPS N.V., EINDHOVEN (NL)

(72) Inventors: Roland Wilhelmus Maria BULLENS, Eindhoven (NL); Markus Johannes Harmen DEN HARTOG, Eindhoven (NL); Javier OLIVAN BESCOS, Eindhoven (NL); Willem-Jan SPOEL, Eindhoven (NL); Martijn Anne VAN LAVIEREN, Eindhoven (NL); Thijs ELENBAAS, Eindhoven (NL)

(21) Appl. No.: 16/124,891

(22) Filed: Sep. 7, 2018

Related U.S. Application Data
(60) Provisional application No. 62/555,550, filed on Sep. 7, 2017.

(51) Int. Cl. A61B 5/0215; A61B 5/00; A61B 6/12; A61B 6/00

(52) U.S. Cl. CPC A61B 5/02156; A61B 5/02158; A61B 5/6851; A61M 2025/0002; A61B 6/12; A61B 6/463; A61B 5/6852

(57) ABSTRACT

Devices, systems, and methods for evaluating a physiological condition of a vessel are disclosed. In an embodiment, a medical system is disclosed. One embodiment of the medical system comprises a medical processing unit in communication with a first pressure sensor, a second pressure sensor, and a radiographic imaging source configured to obtain radiographic images of at least one intravascular instrument positioned within a body lumen. The medical processing unit is configured to: receive the radiographic images obtained by the radiographic imaging source; detect, using the radiographic images, when the first pressure sensor is in a pre-determined orientation with respect to the second pressure sensor; and automatically initiate normalization of the first pressure sensor and the second pressure sensor in response to detecting that the first pressure sensor is in the pre-determined orientation with respect to the second pressure sensor.

### TECHNICAL FIELD

The present disclosure relates generally to the field of intravascular medical devices used to assess the severity of a blockage or other restriction to the flow of fluid, such as blood, through a vessel. Aspects of the present disclosure include automatic initiation of normalization between pressure sensors based on alignment of intravascular devices in radiographic images of the vessel.

### BACKGROUND

Heart disease is a serious health condition affecting millions of people worldwide. One major cause of heart disease is the presence of flow reducing blockages or lesions within blood vessels. For example, accumulation of plaque inside blood vessels can eventually cause occlusion of the blood vessels through the formation of a partial or even a complete blockage. The formation of such blockages can be life-threatening, and surgical intervention is often required to save the lives of afflicted individuals. Common treatment options available to open up an occluded vessel include balloon angioplasty, rotational atherectomy, and placement of intravascular stents.

Currently accepted techniques for assessing the severity of a stenosis in a blood vessel, including ischemia causing lesions, include fractional flow reserve (FFR) and iFR (instant wave-free ratio). FFR is a calculation of the ratio of a distal pressure measurement (taken on the distal side of the stenosis) relative to a proximal pressure measurement (taken on the proximal side of the stenosis). iFR is a calculation of the ratio of a distal pressure measurement relative to a proximal pressure measurement using pressure measurements obtained during a diagnostic window of heartbeat/cardiac cycle when resistance is naturally constant and minimized. FFR and iFR provide an index of stenosis severity that allows determination as to whether the blockage limits blood flow within the vessel to an extent that treatment is required.

In order to ensure that the FFR or iFR is accurately calculated, a physician currently manually initiates a normalization process after the physician determines that the proximal and distal pressure measuring devices are positioned to measure pressure at the same location. In some instances, the physician can forget to normalize the pressure measuring devices, in which case the calculated ratios are inaccurate and do not accurately represent the degree of stenosis severity. The determination that the pressure measuring devices are aligned and/or to start the normalization process are manual steps that slow down and make the workflow more inefficient.

Given the severity and widespread occurrence of heart disease, there remains a need for improved devices, systems, and methods for assessing the severity of a blockage in a vessel and, in particular, a stenosis in a blood vessel. The devices, systems, and associated methods of the present disclosure overcome one or more of the shortcomings of the prior art.

### SUMMARY

The present disclosure is directed to devices, systems, and methods for vascular assessment. A computing system is configured to automatically initiate a normalization procedure for two pressure sensors. Normalizing the two pressure sensors ensures that they detect the same pressure at the same location. An x-ray imaging source obtains x-ray images of one or more intravascular devices positioned within the body of the patient. Based on the x-ray images, the computing system can automatically initiate normalization when the two pressure sensors are in a pre-determined orientation relative to one another. For example, the two pressure sensors can be aligned or one pressure sensor can be aligned with a pressure sensing location of the second pressure sensor. Tracking the location of the one or more intravascular devices using x-ray images and automatically initiating normalization based on the x-ray images advantageously makes a doctor's workflow more efficient. Improvements in diagnosis and/or treatment can also be facilitated because computing system ensures that the normalization step is started such that the pressure sensors detect accurate pressures within the patient's blood vessels.

For example, in an embodiment, a medical system is disclosed that comprises a medical processing unit in communication with a first pressure sensor, a second pressure sensor, and a radiographic imaging source configured to obtain radiographic images of at least one intravascular instrument positioned within a body lumen, wherein the medical processing unit is configured to: receive the radiographic images obtained by the radiographic imaging source; detect, using the radiographic images, when the first pressure sensor is in a pre-determined orientation with respect to the second pressure sensor; and automatically initiate normalization of the first pressure sensor and the second pressure sensor in response to detecting that the first pressure sensor is in the pre-determined orientation with respect to the second pressure sensor.

In one aspect, the system further includes a first intravascular instrument, wherein the first pressure sensor is configured to measure a pressure at a distal portion of the first intravascular instrument. In one aspect, the first pressure sensor is disposed at the distal portion of the first intravascular instrument. In one aspect, the system further includes a second intravascular instrument, wherein the second pressure sensor is configured to measure a pressure at a distal portion of the second intravascular instrument. In one aspect, the second pressure sensor is disposed at a proximal portion of the second intravascular instrument and in communication with a pressure-sensing location at a distal portion of the second intravascular instrument. In one aspect, the medical processing unit is configured to detect when the first and second pressure sensors are in the pre-determined orientation by determining when the first pressure sensor is aligned with the pressure sensing location of the second intravascular instrument. In one aspect, pressure sensing location of the second intravascular instrument comprises an ostium at the distal portion. In one aspect, the medical processing unit is configured to detect when the first and second pressure sensors are in the pre-determined orientation by determining when the first pressure sensor is aligned with the ostium of the second intravascular instrument. In one aspect, the second intravascular instrument comprises a catheter. In one aspect, the first intravascular instrument comprises a guide wire.

In one aspect, the medical processing unit is configured to detect when the first and second pressure sensors are in the pre-determined orientation based on a radiopaque region of at least one of the first or second intravascular instruments. In one aspect, the medical processing unit is further configured to track locations of the first intravascular instrument and second intravascular instrument within the body lumen while at least one of the first or second intravascular instruments is being moved through the body lumen. In one aspect, the medical processing unit is further configured to prompt an operator when the first and second sensors are nearing the pre-determined orientation. In one aspect, the medical processing unit is further configured to prompt an operator to refrain from moving the first and second intravascular instruments in response to detecting that the first pressure sensor is in the pre-determined orientation with respect to the second pressure sensor. In one aspect, the medical processing unit is further configured to display the radiographic images and to visually enhance depiction of the first and second intravascular instruments in the displayed radiographic images. In one aspect, the medical processing unit is configured to visually enhance depiction of the first and second intravascular instruments by highlighting the first intravascular instrument with a first color and highlighting the second intravascular instrument with a second color.

In another embodiment, a method is disclosed that comprises receiving, by a medical processing unit in communication with a radiographic imaging source, radiographic images of at least one intravascular instrument obtained by the radiographic imaging source; detecting, by the medical processing unit, when a first pressure sensor is in a pre-determined orientation with respect to a second pressure sensor based on the radiographic images; and automatically initiating normalization of the first pressure sensor and the second pressure sensor in response to detecting that the first pressure sensor is in the pre-determined orientation with respect to the second pressure sensor.

In one aspect, the at least one intravascular instrument comprises a first intravascular instrument and a second intravascular instrument, the first pressure sensor associated with the first intravascular instrument and the second pressure sensor associated with the second intravascular instrument, and wherein the detecting comprises: determining when the first pressure sensor is aligned with a pressure sensing location of the second intravascular instrument, the second pressure sensor in communication with the pressure sensing location. In one aspect, the method further includes outputting, by the medical processing unit, data representative of a prompt to prompt an operator to refrain from moving the first and second intravascular instruments in response to detecting that the first pressure sensor is in the pre-determined orientation with respect to the second pressure sensor. In one aspect, the method further includes displaying the radiographic images and visually enhancing depiction of the first and second intravascular instruments in the displayed radiographic images.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Illustrative embodiments of the present disclosure will be described with reference to the accompanying drawings, of which:

FIG. 1 is a diagrammatic perspective view of a vessel having a stenosis according to an embodiment of the present disclosure.

FIG. 2 is a diagrammatic, partial cross-sectional perspective view of a portion of the vessel of FIG. 1 taken along section line 2-2 of FIG. 1.

FIG. 3 is a diagrammatic, partial cross-sectional perspective view of the vessel of FIG. 1 and FIG. 2 with instruments positioned therein according to an embodiment of the present disclosure.

FIG. 4 is a diagrammatic, schematic view of a system according to an embodiment of the present disclosure.

FIG. 5 is a diagrammatic, schematic side view of a plurality of intravascular instruments according to an embodiment of the present disclosure.

FIG. 6 is a diagrammatic, schematic side view of a plurality of intravascular instruments according to an embodiment of the present disclosure.

FIG. 7 is a diagrammatic, schematic side view of an intravascular instrument according to an embodiment of the present disclosure.

FIG. 8A is a radiographic image of a plurality of intravascular instruments according to an embodiment of the present disclosure.

FIG. 8B is a radiographic image of a plurality of intravascular instruments according to an embodiment of the present disclosure.

FIG. 9 is a flowchart of a method according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, the numerous iterations of these combinations will not be described separately.

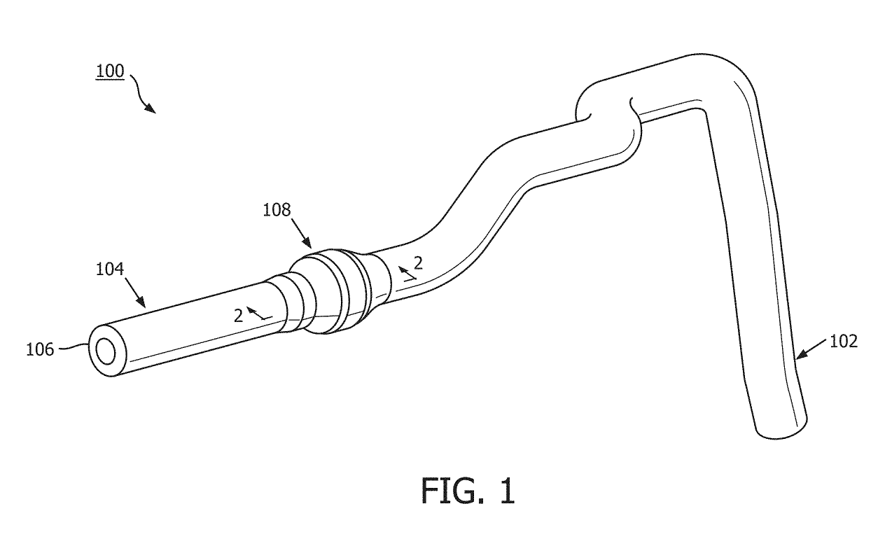

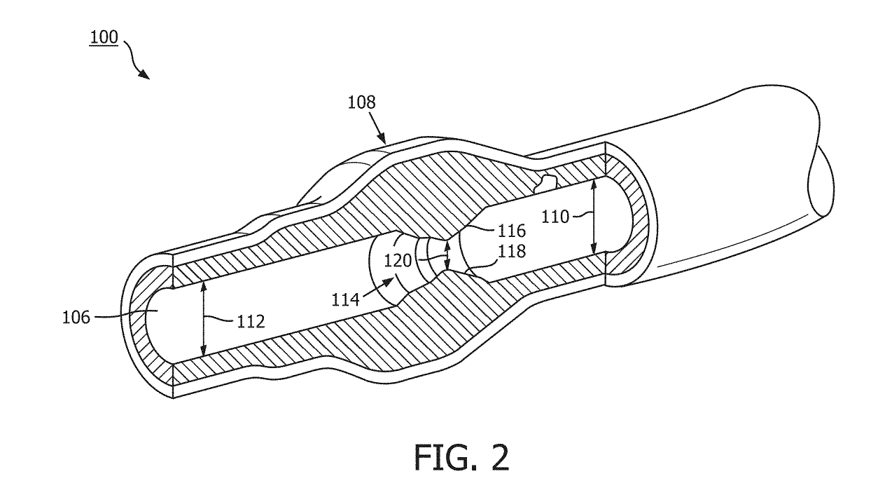

Referring to FIG. 1 and FIG. 2, shown therein is a vessel 100 having a stenosis according to an embodiment of the present disclosure. In that regard, FIG. 1 is a diagrammatic perspective view of the vessel 100, and FIG. 2 is a partial cross-sectional perspective view of a portion of the vessel 100 taken along section line 2-2 of FIG. 1. Referring more specifically to FIG. 1, the vessel 100 includes a proximal portion 102 and a distal portion 104. A lumen 106 extends along the length of the vessel 100 between the proximal portion 102 and the distal portion 104. The lumen 106 is configured to allow the flow of fluid through the vessel. In some instances, the vessel 100 is a systemic blood vessel. In some particular instances, the vessel 100 is a coronary artery. In such instances, the lumen 106 is configured to facilitate the flow of blood through the vessel 100.

As shown, the vessel 100 includes a stenosis 108 between the proximal portion 102 and the distal portion 104. Stenosis 108 is generally representative of any blockage or other structural arrangement that results in a restriction to the flow of fluid through the lumen 106 of the vessel 100. Embodiments of the present disclosure are suitable for use in a wide variety of vascular applications, including without limitation coronary, peripheral (including but not limited to lower limb, carotid, and neurovascular), renal, and/or venous. Where the vessel 100 is a blood vessel, the stenosis 108 may be a result of plaque buildup, including without limitation plaque components such as fibrous, fibro-lipidic (fibro fatty), necrotic core, calcified (dense calcium), blood, fresh thrombus, and mature thrombus. Generally, the composition of the stenosis will depend on the type of vessel being evaluated. It is understood that the concepts of the present disclosure are applicable to virtually any type of blockage or other narrowing of a vessel that results in decreased fluid flow.

Referring more particularly to FIG. 2, the lumen 106 of the vessel 100 has a diameter 110 proximal of the stenosis 108 and a diameter 112 distal of the stenosis 108. In some instances, the diameters 110 and 112 may be substantially equal to one another. In that regard, the diameters 110 and 112 are intended to represent healthy portions, or at least healthier portions, of the lumen 106 in comparison to stenosis 108. Accordingly, these healthier portions of the lumen 106 are illustrated as having a substantially constant cylindrical profile and, as a result, the height or width of the lumen has been referred to as a diameter. However, it is understood that in many instances these portions of the lumen 106 will also have plaque buildup, a non-symmetric profile, and/or other irregularities, but to a lesser extent than stenosis 108 and, therefore, will not have a cylindrical profile. In such instances, the diameters 110 and 112 are understood to be representative of a relative size or cross-sectional area of the lumen and do not imply a circular cross-sectional profile.

As shown in FIG. 2, stenosis 108 includes plaque buildup 114 that narrows the lumen 106 of the vessel 100. In some instances, the plaque buildup 114 may not have a uniform or symmetrical profile, making angiographic evaluation of such a stenosis potentially unreliable. In the illustrated embodiment, the plaque buildup 114 includes an upper portion 116 and an opposing lower portion 118. The lower portion 118 has an increased thickness relative to the upper portion 116 that results in a non-symmetrical and non-uniform profile relative to the portions of the lumen proximal and distal of the stenosis 108. As shown, the plaque buildup 114 decreases the available space for fluid to flow through the lumen 106. In particular, the cross-sectional area of the lumen 106 is decreased by the plaque buildup 114. At the narrowest point between the upper and lower portions 116, 118 the lumen 106 has a height 120, which is representative of a reduced size or cross-sectional area relative to the diameters 110 and 112 proximal and distal of the stenosis 108. Note that the stenosis 108, including plaque buildup 114, is exemplary in nature and should not be considered limiting in any way. In that regard, it is understood that the stenosis 108 has other shapes and/or compositions that limit the flow of fluid through the lumen 106 in other instances. While the vessel 100 is illustrated in FIG. 1 and FIG. 2 as having a single stenosis 108 and the description of the embodiments below is primarily made in the context of a single stenosis, it is nevertheless understood that the devices, systems, and methods described herein have similar application for a vessel having multiple stenosis regions.

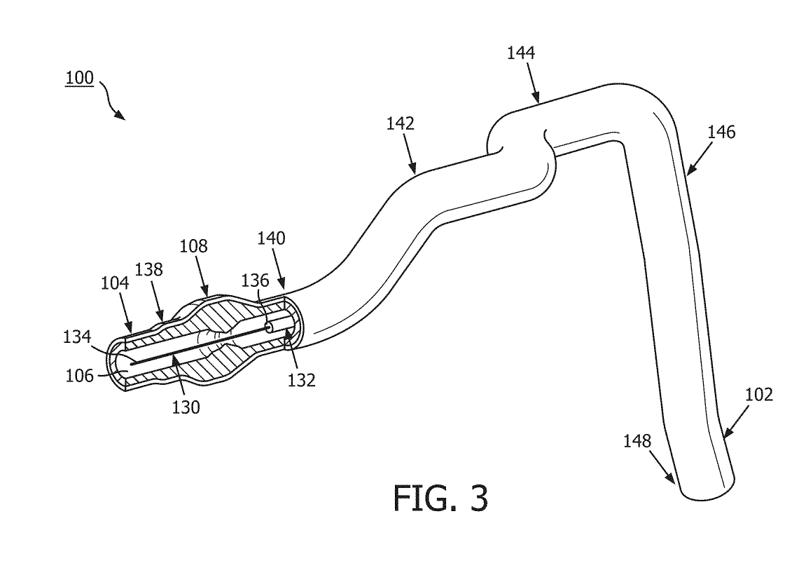

Referring now to FIG. 3, the vessel 100 is shown with instruments 130 and 132 positioned therein according to an embodiment of the present disclosure. In general, instruments 130 and 132 may comprise any form of device, instrument, or probe sized and shaped to be positioned within a vessel. In the illustrated embodiment, instrument 130 is generally representative of a guide wire, and instrument 132 is generally representative of a catheter or guide catheter. Generally, the instruments 130, 132 can include a flexible elongate member including a proximal portion and a distal portion. In that regard, instrument 130 may extend through a central lumen of instrument 132. However, in other embodiments, the instruments 130 and 132 may take other forms. The instruments 130 and 132 may take similar form in some embodiments. For example, in some instances, both instruments 130 and 132 may comprise guide wires. In other instances, both instruments 130 and 132 may comprise catheters. On the other hand, the instruments 130 and 132 may take different forms in some embodiments, such as the illustrated embodiment, where one of the instruments comprises a catheter and the other a guide wire. Further, in some instances, the instruments 130 and 132 may be disposed coaxial with one another, as shown in the illustrated embodiment of FIG. 3. In other instances, one of the instruments may extend through an off-center lumen of the other instrument. In yet other instances, the instruments 130 and 132 may extend side-by-side. In some particular embodiments, at least one of the instruments may comprise a rapid-exchange device, such as a rapid-exchange catheter. In such embodiments, the other instrument may comprise a buddy wire or other device configured to facilitate the introduction and removal of the rapid-exchange device. Further still, in other instances, a single instrument may be utilized instead of two separate instruments 130 and 132. In that regard, the single instrument may incorporate aspects of the functionalities (e.g., data acquisition) of both instruments 130 and 132 in some embodiments.

Instrument 130 may be configured to obtain diagnostic information about the vessel 100. While, in some contexts, diagnostic information may comprise diagnostic data, biological information, biological data, cardiovascular information, cardiovascular data, and/or other information or data, for the purposes of the present disclosure, the term “diagnostic information” will be used. Diagnostic information may be gathered continuously, approximately every 0.01 seconds, approximately every 0.1 seconds, approximately every 0.25 seconds, approximately every 0.5 seconds, approximately once per second, approximately once every two seconds, approximately once every 5 seconds, approximately once every 10 seconds, approximately once per heartbeat, and/or over some other timeframe. It is also contemplated that diagnostic information may be gathered in response to a trigger, in response to a command, or in response to a request. The diagnostic information may include one or more of pressure, flow (velocity), images (including images obtained using ultrasound (e.g., IVUS), OCT, thermal, and/or other imaging techniques), temperature, heart rate, electrical activity, and/or combinations thereof.

Accordingly, the instrument 130 may include one or more sensors, transducers, and/or other monitoring elements configured to obtain the diagnostic information about the vessel. The one or more sensors, transducers, and/or other monitoring elements may be positioned adjacent a distal portion of the instrument 130. The sensors, transducers, and/or other monitoring elements may be described with reference to an aspect of their implementation. For example, a pressure sensor may comprise a sensor configured to measure a pressure. In another example, an aortic transducer may comprise a transducer located in the aorta and/or interacting with diagnostic information pertaining to the aorta. In some instances, the transducer, such as an aortic transducer, can be positioned outside of the patient body and/or at a proximal portion of the instrument 130. For example, the transducer can be in fluid communication with a pressure sensing location at a distal portion of instrument 130 that is positioned within the patient body. The pressure at the pressure sensing location within patient body can be measured by the aortic transducer based on the fluid communication. In some instances, the one or more sensors, transducers, and/or other monitoring elements may be positioned less than 30 cm, less than 10 cm, less than 5 cm, less than 3 cm, less than 2 cm, and/or less than 1 cm from a distal tip 134 of the instrument 130. In an embodiment, at least one of the one or more sensors, transducers, and/or other monitoring elements may be positioned at the distal tip of the instrument 130.

The instrument 130 may include at least one element configured to monitor pressure within the vessel 100. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element may be implemented as a solid-state component manufactured using semiconductor and/or other suitable manufacturing techniques. Examples of commercially available guide wire products that include suitable pressure monitoring elements include, without limitation, the PrimeWire PRESTIGE® pressure guide wire, the PrimeWire® pressure guide wire, and the ComboWire® XT pressure and flow guide wire, each available from Volcano Corporation, as well as the PressureWire™ Certus guide wire and the PressureWire™ Aeris guide wire, each available from St. Jude Medical, Inc. The instrument 130 may be sized such that it can be positioned through the stenosis 108 without significantly impacting fluid flow across the stenosis, which would impact the distal pressure reading. Accordingly, in some instances the instrument 130 may have an outer diameter of 0.018″ or less. In some embodiments, the instrument 130 may have an outer diameter of 0.014″ or less.

Instrument 132 may also be configured to obtain diagnostic information about the vessel 100. In some instances, instrument 132 may be configured to obtain the same diagnostic information as instrument 130. In other instances, instrument 132 may be configured to obtain different diagnostic information than instrument 130, which may include additional diagnostic information, less diagnostic information, and/or alternative diagnostic information. Diagnostic information may be gathered continuously, approximately every 0.01 seconds, approximately every 0.1 seconds, approximately every 0.25 seconds, approximately every 0.5 seconds, approximately once per second, approximately once every two seconds, approximately once every 5 seconds, approximately once every 10 seconds, approximately once per heartbeat, and/or over some other timeframe. It is also contemplated that diagnostic information may be gathered in response to a trigger, in response to a command, and/or in response to a request. The diagnostic information obtained by instrument 132 may include one or more of pressure, flow (velocity), images (including images obtained using ultrasound (e.g., IVUS), OCT, thermal, and/or other imaging techniques), temperature, or combinations thereof.

Instrument 132 may include one or more sensors, transducers, and/or other monitoring elements configured to obtain this diagnostic information. In an embodiment, the one or more sensors, transducers, and/or other monitoring elements may be positioned adjacent a distal portion of the instrument 132. The sensors, transducers, and/or other monitoring elements may be described with reference to an aspect of their implementation. For example, a pressure sensor may comprise a sensor configured to measure a pressure. In another example, an aortic transducer may comprise a transducer located in the aorta and/or interacting with diagnostic information pertaining to the aorta. In some instances, the transducer, such as an aortic transducer, can be positioned outside of the patient body and/or at a proximal portion of the instrument 130. For example, the transducer can be in fluid communication with a pressure sensing location at a distal portion of instrument 132 that is positioned within the patient body. The pressure at the pressure sensing location within patient body can be measured by the aortic transducer based on the fluid communication. The one or more sensors, transducers, and/or other monitoring elements may be positioned less than 30 cm, less than 10 cm, less than 5 cm, less than 3 cm, less than 2 cm, and/or less than 1 cm from a distal tip 136 of the instrument 132. In some instances, at least one of the one or more sensors, transducers, and/or other monitoring elements may be positioned at the distal tip of the instrument 132.

Similar to instrument 130, instrument 132 may also include at least one element configured to monitor pressure within the vessel 100. The pressure monitoring element can take the form a piezo-resistive pressure sensor, a piezo-electric pressure sensor, a capacitive pressure sensor, an electromagnetic pressure sensor, a fluid column (the fluid column being in communication with a fluid column sensor that is separate from the instrument and/or positioned at a portion of the instrument proximal of the fluid column), an optical pressure sensor, and/or combinations thereof. In some instances, one or more features of the pressure monitoring element may be implemented as a solid-state component manufactured using semiconductor and/or other suitable manufacturing techniques. Millar catheters may be utilized in some embodiments. Currently available catheter products suitable for use with one or more of Philips's Xper Flex Cardio Physiomonitoring System, GE's Mac-Lab XT and XTi hemodynamic recording systems, Siemens's AXIOM Sensis XP VC11, McKesson's Horizon Cardiology Hemo, and Mennen's Horizon XVu Hemodynamic Monitoring System and include pressure monitoring elements can be utilized for instrument 132 in some instances.

In accordance with aspects of the present disclosure, at least one of the instruments 130 and 132 may be configured to monitor a pressure, e.g., a blood pressure, within the vessel 100 distal of the stenosis 108 and at least one of the instruments 130 and 132 may be configured to monitor a pressure within the vessel proximal of the stenosis. In that regard, the instruments 130, 132 may be sized and shaped to allow positioning of the at least one element configured to monitor pressure within the vessel 100 to be positioned proximal and/or distal of the stenosis 108 as appropriate based on the configuration of the devices. FIG. 3 illustrates a position 138 suitable for measuring pressure distal of the stenosis 108. The position 138 may be less than 5 cm, less than 3 cm, less than 2 cm, less than 1 cm, less than 5 mm, and/or less than 2.5 mm from the distal end of the stenosis 108 (as shown in FIG. 2) in some instances.

FIG. 3 also illustrates a plurality of suitable positions for measuring pressure proximal of the stenosis 108. Positions 140, 142, 144, 146, and 148 each represent a position that may be suitable for monitoring the pressure proximal of the stenosis in some instances. The positions 140, 142, 144, 146, and 148 are positioned at varying distances from the proximal end of the stenosis 108 ranging from more than 20 cm down to about 5 mm or less. The proximal pressure measurement can be spaced from the proximal end of the stenosis. Accordingly, in some instances, the proximal pressure measurement may be taken at a distance equal to or greater than an inner diameter of the lumen of the vessel from the proximal end of the stenosis. In the context of coronary artery pressure measurements, the proximal pressure measurement may be taken at a position proximal of the stenosis and distal of the aorta, within a proximal portion of the vessel. However, in some particular instances of coronary artery pressure measurements, the proximal pressure measurement may be taken from a location inside the aorta. In such instances, the pressure data obtained may be referred to as aortic pressure data. In other instances, the proximal pressure measurement may be taken at the root or ostium of the coronary artery.

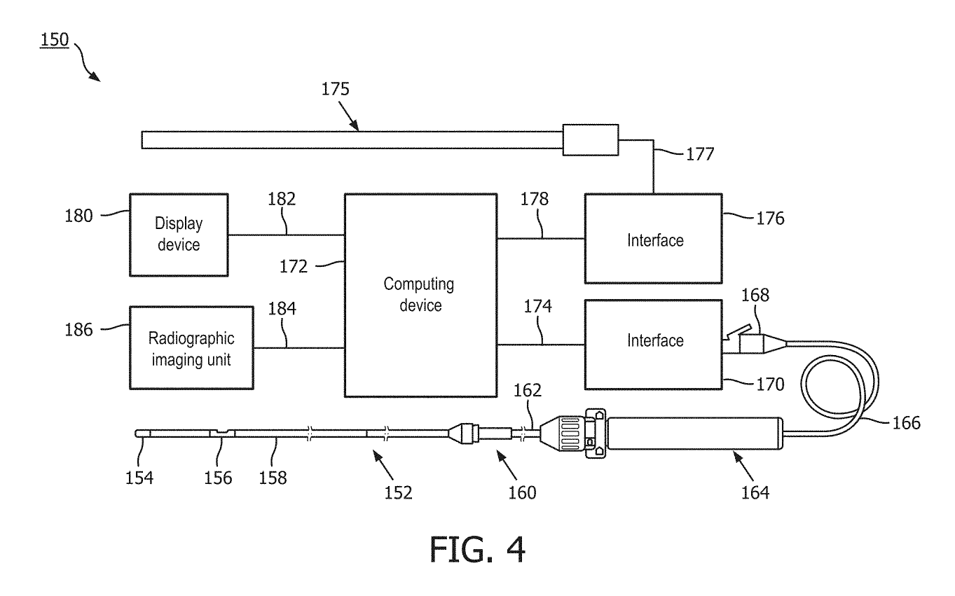

Referring now to FIG. 4, shown therein is a system 150 according to an embodiment of the present disclosure. In that regard, FIG. 4 is a diagrammatic, schematic view of the system 150. As shown, the system 150 includes an instrument 152. In that regard, in some instances instrument 152 is suitable for use as at least one of instruments 130 and 132 discussed above. Accordingly, in some instances the instrument 152 includes features similar to those discussed above with respect to one or both of instruments 130 and 132. In the illustrated embodiment, the instrument 152 is a guide wire having a distal portion 154 and a housing 156 positioned adjacent the distal portion. In that regard, the housing 156 is spaced approximately 3 cm from a distal tip of the instrument 152. The housing 156 is configured to house one or more sensors, transducers, and/or other monitoring elements configured to obtain the diagnostic information about the vessel. In the illustrated embodiment, the housing 156 contains at least a pressure sensor configured to monitor a pressure within a lumen in which the instrument 152 is positioned. A shaft 158 extends proximally from the housing 156. A torque device 160 is positioned over and coupled to a proximal portion of the shaft 158. A proximal end portion 162 of the instrument 152 is coupled to a connector 164. A cable 166 extends from connector 164 to a connector 168. In some instances, connector 168 is configured to be plugged into an interface 170. In that regard, interface 170 is a patient interface module (PIM) in some instances. The cable 166 may be replaced with a wireless connection in some instances. In that regard, it is understood that various communication pathways between the instrument 152 and the interface 170 may be utilized, including physical connections (including electrical, optical, and/or fluid connections), wireless connections, and/or combinations thereof.

The interface 170 is communicatively coupled to a computing device 172 via a connection 174. Computing device 172 is generally representative of any device suitable for performing the processing and analysis techniques discussed within the present disclosure. In some embodiments, the computing device 172 includes a processor, random access memory, and a storage medium. In that regard, in some particular instances the computing device 172 is programmed to execute steps associated with the normalization, data acquisition, and data analysis described herein. Accordingly, it is understood that any steps related to normalization, data acquisition, data processing, instrument control, and/or other processing or control aspects of the present disclosure may be implemented by the computing device 172 using corresponding instructions stored on or in a non-transitory computer readable medium accessible by the computing device 172. In some instances, the computing device 172 is a console device. In some particular instances, the computing device 172 is similar to the s5 Imaging System or the s5i Imaging System, each available from Volcano Corporation. In some instances, the computing device 172 is portable (e.g., handheld, on a rolling cart, etc.). In some instances, all or a portion of the computing device 172 can be implemented as a bedside controller such that one or more processing steps described herein can be performed by processing component(s) of the bedside controller. An exemplary bedside controller is described in U.S. Provisional Application No. 62/049,265, titled “Bedside Controller for Assessment of Vessels and Associated Devices, Systems, and Methods,” and filed Sep. 11, 2014, the entirety of which is hereby incorporated by reference. Further, it is understood that in some instances the computing device 172 comprises a plurality of computing devices and/or virtual computing devices. In that regard, it is particularly understood that the different processing and/or control aspects of the present disclosure may be implemented separately or within predefined groupings using a plurality of computing devices and/or virtual computing devices. Any divisions and/or combinations of the processing and/or control aspects described below across multiple computing devices and/or virtual computing devices are within the scope of the present disclosure.

Together, connector 164, cable 166, connector 168, interface 170, and connection 174 facilitate communication between the one or more sensors, transducers, and/or other monitoring elements of the instrument 152 and the computing device 172. However, this communication pathway is exemplary in nature and should not be considered limiting in any way. In that regard, it is understood that any communication pathway between the instrument 152 and the computing device 172 may be utilized, including physical connections (including electrical, optical, and/or fluid connections), wireless connections, and/or combinations thereof. In that regard, it is understood that the connection 174 is wireless in some instances. In some instances, the connection 174 includes a communication link over a network (e.g., intranet, internet, telecommunications network, and/or other network). In that regard, it is understood that the computing device 172 is positioned remote from an operating area where the instrument 152 is being used in some instances. Having the connection 174 include a connection over a network can facilitate communication between the instrument 152 and the remote computing device 172 regardless of whether the computing device is in an adjacent room, an adjacent building, or in a different state/country. Further, it is understood that the communication pathway between the instrument 152 and the computing device 172 is a secure connection in some instances. Further still, it is understood that, in some instances, the data communicated over one or more portions of the communication pathway between the instrument 152 and the computing device 172 is encrypted.

The system 150 also includes an instrument 175. In that regard, in some instances instrument 175 is suitable for use as at least one of instruments 130 and 132 discussed above. Accordingly, in some instances the instrument 175 includes features similar to those discussed above with respect to one or both of instruments 130 and 132. In the illustrated embodiment, the instrument 175 is a catheter-type device. In that regard, the instrument 175 includes one or more sensors, transducers, and/or other monitoring elements adjacent a distal portion of the instrument 175 configured to obtain the diagnostic information about the vessel. In the illustrated embodiment, the instrument 175 includes a pressure sensor configured to monitor a pressure within a lumen in which the instrument 175 is positioned. The instrument 175 is in communication with an interface 176 via connection 177. In some instances, interface 176 is a hemodynamic monitoring system or other control device, such as Siemens AXIOM Sensis, Mennen Horizon XVu, and Philips Xper IM Physiomonitoring 5. In one particular embodiment, instrument 175 is a pressure-sensing catheter that includes fluid column extending along its length. In such an embodiment, interface 176 includes a hemostasis valve fluidly coupled to the fluid column of the catheter, a manifold fluidly coupled to the hemostasis valve, and tubing extending between the components as appropriate to fluidly couple the components. In that regard, the fluid column of the catheter is in fluid communication with a pressure sensor via the valve, manifold, and tubing. In some instances, the pressure sensor is part of interface 176. In other instances, the pressure sensor is a separate component positioned between the instrument 175 and the interface 176. The interface 176 is communicatively coupled to the computing device 172 via a connection 178.

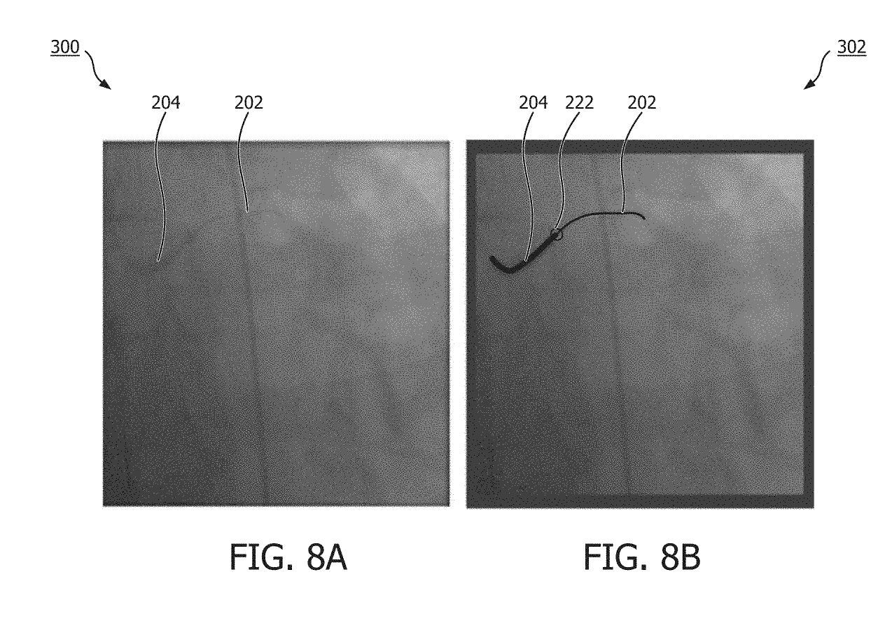

The computing device 172 is communicatively coupled to a display device 180 via a wired or wireless connection 182. In some embodiments, the display device 180 is a component of the computing device 172, while in other embodiments, the display device 180 is distinct from the computing device 172. In some embodiments, the display device 180 is implemented as a bedside controller having a touch-screen display as described, for example, in U.S. Provisional Application No. 62/049,265, titled “Bedside Controller for Assessment of Vessels and Associated Devices, Systems, and Methods,” and filed Sep. 11, 2014, the entirety of which is hereby incorporated by reference herein. The computing device 172 can generate screen displays including data collected by the instruments 152 and 175 and other instruments, quantities computed based on the collected data, visualizations of the vessel in which the data is collected, and visualizations based on the collected data and computed quantities. Exemplary screen displays are illustrated in FIGS. 8A and 8B. The computing device 172 can provide the display data associated with the screen displays to the display device 180.

The computing device 172 is communicatively coupled to a radiographic unit 186. For example, data obtained by the radiographic unit 186 can be directly or indirectly transmitted to and/or received by the computing device 172, e.g., via a wired or wireless connection 184. The radiographic unit 186 may obtain diagnostic information of a patient's vasculature and may communicate such diagnostic information to the computing device 172. In various embodiments, the diagnostic information obtained by the radiographic unit 186 may include externally-obtained angiographic images, x-ray images, CT images, PET images, MRI images, SPECT images, fluoroscopic images, radiographic images, combinations thereof and/or other two-dimensional or three-dimensional extraluminal depictions of a patient's vasculature. For example, angiographic images can be single frame, still, radiographic images of the vasculature of the patient and/or one or more intravascular devices positioned within the vasculature. For example, fluoroscopic images can be multi-frame, moving radiographic images of the vasculature and/or one or more intravascular devices positioned within the vasculature. In some cases, diagnostic information and/or data obtained by instruments 130, 132, 152, and/or 175 may be correlated or co-registered to diagnostic information such as angiographic image(s) and/or other two-dimensional or three-dimensional depictions of a patient's vasculature obtained by the radiographic unit 186. In some embodiments, the radiographic unit 186 obtains radiographic images after contrast media has been delivered into the vessel and/or other lumen. In other embodiments, the radiographic unit 186 obtains radiographic images without contrast media within the vessel and/or other lumen.

The computing device 172 can additionally be communicatively coupled to a user interface device. The user interface device permits a user to interact with the screen displays on the display device 180. For example, the user can provide a user input to modify all or a portion of the screen display using the user interface device. In some embodiments, the user interface device is a separate component from the display device 180. In other embodiments, the user interface device is part of the display device 180. For example, the user interface device can be implemented as a bedside controller having a touch-screen display as described, for example, in U.S. Provisional Application No. 62/049,265, titled “Bedside Controller for Assessment of Vessels and Associated Devices, Systems, and Methods,” and filed Sep. 11, 2014, the entirety of which is hereby incorporated by reference herein. In such embodiments, a user input can be a touch input received on the touch sensitive display of the bedside controller.

Similar to the connections between instrument 152 and the computing device 172, interface 176 and connections 177 and 178 facilitate communication between the one or more sensors, transducers, and/or other monitoring elements of the instrument 175 and the computing device 172. However, this communication pathway is exemplary in nature and should not be considered limiting in any way. In that regard, it is understood that any communication pathway between the instrument 175 and the computing device 172 may be utilized, including physical connections (including electrical, optical, and/or fluid connections), wireless connections, and/or combinations thereof. In that regard, it is understood that the connection 178 is wireless in some instances. In some instances, the connection 178 includes a communication link over a network (e.g., intranet, internet, telecommunications network, and/or other network). In that regard, it is understood that the computing device 172 is positioned remote from an operating area where the instrument 175 is being used in some instances. Having the connection 178 include a connection over a network can facilitate communication between the instrument 175 and the remote computing device 172 regardless of whether the computing device is in an adjacent room, an adjacent building, or in a different state/country. Further, it is understood that the communication pathway between the instrument 175 and the computing device 172 is a secure connection in some instances. Further still, it is understood that, in some instances, the data communicated over one or more portions of the communication pathway between the instrument 175 and the computing device 172 is encrypted.

It is understood that one or more components of the system 150 are not included, are implemented in a different arrangement/order, and/or are replaced with an alternative device/mechanism in other embodiments of the present disclosure. For example, in some instances, the system 150 does not include interface 170 and/or interface 176. In such instances, the connector 168 (or other similar connector in communication with instrument 152 or instrument 175) may plug into a port associated with computing device 172. Alternatively, the instruments 152, 175 may communicate wirelessly with the computing device 172. Generally speaking, the communication pathway between either or both of the instruments 152, 175 and the computing device 172 may have no intermediate nodes (i.e., a direct connection), one intermediate node between the instrument and the computing device, or a plurality of intermediate nodes between the instrument and the computing device.

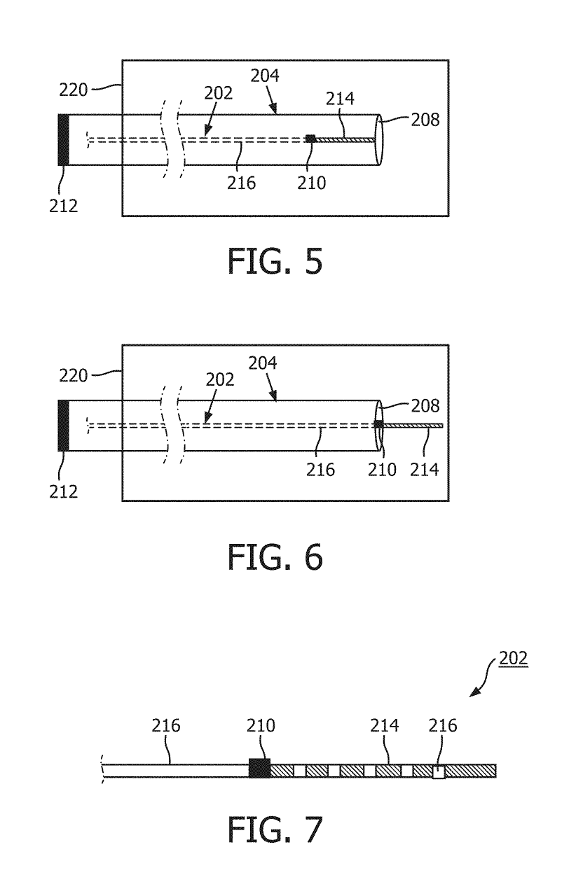

Referring now to FIGS. 5 and 6, shown therein are a diagrammatic, schematic side views of a plurality of intravascular instruments, intravascular instrument 202 and intravascular instrument 204. FIGS. 5 and 6 illustrate distal portions of the intravascular instruments 202, 204. It is understand that, in use, the intravascular instruments 202, 204 are positioned within vasculature of a patient. In an embodiment, one or both of intravascular instruments 202 and 204 may be elements of the system 150 and/or may interact with elements of the system 150. In that regard, in some instances, intravascular instrument 202 may be suitable for use as at least one of instruments 130, 132, 152, and 175 discussed above. Accordingly, in some instances the intravascular instrument 202 includes features similar to those discussed above with respect to one or more of instruments 130, 132, 152, and 175. Similarly, in some instances, intravascular instrument 204 may be suitable for use as at least one of instruments 130, 132, 152, and 175 discussed above and may include features similar to those discussed above with respect to one or more of instruments 130, 132, 152, and 175. In some cases, the intravascular instrument 202 may comprise a pressure-sensing guide wire, and the intravascular instrument 204 may comprise a pressure-sensing catheter or a guide catheter.

In the illustrated embodiment, the intravascular instrument 202 is disposed within a lumen of intravascular instrument 204 and comprises a sensor 210, a radiopaque region 214, and a non-radiopaque region 216 while the intravascular instrument 204 comprises a pressure sensing location 208. The sensor 210 may be configured to obtain pressure data. In that regard, the sensor 210 and/or the pressure sensing location 208 may comprise a piezo-resistive pressure sensor, a piezo-electric pressure sensor, a capacitive pressure sensor, an electromagnetic pressure sensor, a fluid column (the fluid column being in communication with a fluid column sensor that is separate from the intravascular instruments 202 and 204 and/or positioned at a portion of one or the other or both of intravascular instruments 202 and 204 that is proximal of the fluid column), an optical pressure sensor, and/or combinations thereof. In an embodiment, the pressure sensing location 208 may include one or more apertures marking the beginning of a fluid column through which pressure waves may be transmitted to a pressure sensor or other sensor located external to a patient into whose vasculature the intravascular instrument 204 has been placed.

Intravascular instruments 202 and 204 may be used to gather diagnostic information from inside a patient's vasculature, e.g., inside the aorta and/or coronary arteries. In that regard, intravascular instruments 202 and 204 may gather pressure data, flow (velocity) data, images (including images obtained using ultrasound (e.g., IVUS), OCT, thermal, and/or other imaging techniques), temperature data, or combinations thereof. In some instances, intravascular instruments 202 and 204 may be used to gather pressure data to be used in calculating a pressure ration, a FFR value, and/or an iFR (instant wave-free ratio) value. Exemplary embodiments of determining a diagnostic window within a heartbeat cycle of a patient and calculating an iFR value based on pressure measurements obtained within the diagnostic window are described in U.S. Pat. No. 9,339,348, the entirety of which is hereby incorporated by reference herein. Accordingly, the intravascular instruments 202 and 204 may be arranged within a vessel having a stenosis such that one of the intravascular instruments 202 and 204 may measure pressure distal of the stenosis (Pd) and the other of the intravascular instruments 202 and 204 may measure pressure proximal of the stenosis (Pa). In that regard, the intravascular instrument 202 may be positioned within the vessel such that the sensor 210 is distal to the stenosis while the intravascular instrument 204 is positioned such that the pressure sensing location is proximal to the stenosis or vice versa.

To reduce the risk of error, the pressure sensors used to gather proximal and distal pressure data may be calibrated to each other. For example, the sensor 210 of the intravascular instrument 202 may be calibrated to a sensor of the intravascular instrument 204, which may sense pressure at the pressure sensing location 208, e.g., via a fluid column, or vice versa. Such calibration may be referred to as normalization in some cases, and pressure sensors which have been calibrated to each other may in some instances be referred to as having been normalized. Similarly, intravascular instruments which have had their sensors normalized to each other may be referred to as having been themselves normalized to each other. Normalization may involve calibration of pressure wave amplitudes, phases, frequencies, or combinations thereof. Generally, both the intravascular instruments 202, 204 should measure the same pressure at the same location within the vessel. Normalization allows for the computing device to verify the that both of the intravascular instruments 202, 204 are indeed measuring the same pressure at same location, and if not, adjust the intravascular instruments 202, 204 and/or the pressure signals received from the intravascular instruments 202,204 to ensure that the same pressure is being measured at same location.

For example, for a variety of reasons, proximal pressure data and distal pressure data may not be temporally aligned in some instances. In that regard, during data acquisition, there will often be a delay between the distal pressure measurement signals and the proximal pressure measurement signals due to hardware signal handling differences between the instrument(s), e.g., intravascular instruments 202 and 204, utilized to obtain the measurements. The differences can come from physical sources (such as cable length and/or varying electronics) and/or can be due to signal processing differences (such as filtering techniques). In some embodiments, the proximal pressure measurement signal may be acquired and routed through a hemodynamic monitoring system or other interface and may take significantly longer to reach the processing hardware or computing device compared to the distal pressure measurement signal that may be sent more directly to the processing hardware or computing device. The resulting delay may be between about 5 ms and about 150 ms in some instances. Delays between the proximal and distal pressure measurement signals can have a significant impact on alignment of the pressure data. As a result, in some instances, it may be beneficial to shift one of the proximal and distal pressures relative to the other of the distal and proximal pressures in order to temporally align the pressure measurements.

Alignment of all or portion(s) of the proximal and distal pressures may be accomplished using a hardware approach, a software approach, or some combination of the two. Typically, the pressure values obtained by the pressure sensing instrument utilized for monitoring pressure distal of the stenosis is adjusted to match the pressure values of the pressure sensing instrument utilized for monitoring pressure proximal of the stenosis. However, in other embodiments the pressure values obtained by the pressure sensing instrument utilized for monitoring pressure proximal of the stenosis is adjusted to match the pressure values of the pressure sensing instrument utilized for monitoring pressure distal of the stenosis. In yet other embodiments, the pressure values obtained by the pressure sensing instrument utilized for monitoring pressure proximal of the stenosis and the pressure sensing instrument utilized for monitoring pressure distal of the stenosis are adjusted to match an intermediate pressure measurement value (i.e., a pressure value between that measured by each of the pressure sensing instruments).

In an embodiment, the sensor 210 of the intravascular instrument 202 and a sensor 212 of the intravascular instrument 204, which may sense pressure at the pressure sensing location 208, may be normalized. In order to reduce the effect of pressure variances potentially present at different locations within a patient vasculature into which intravascular instruments 202 and 204 have been positioned, normalization may be performed when the sensors 210, 212 are in a pre-determined spatial or physical orientation with respect to one another. FIGS. 5 and 6 illustrate at least portion (e.g., central and/or distal portions) of the intravascular instruments 202, 204 disposed within the body 220 of a patient, such as within a blood vessel. The pressure sensor 210 of the instrument 202 is disposed at the distal portion of the instrument 202 and positioned within the body 220. The sensor 212 is disposed at the proximal portion of the instrument 204 and positioned outside of the body 220. The sensor 212 can be in communication (e.g., fluid communication via a fluid column) with a pressure sensing location 208 of the instrument 204. Generally, the sensor 212 measures a pressure within the aorta or an ostium of a blood vessel, proximal of a stenosis of the vessel, because the pressure sensing location 208 is positioned at the aorta or the ostium. The sensor 210 measure a pressure within the blood vessel, distally and/or distally of the stenosis of the vessel. According to aspects of the present disclosure, the sensors 210, 212 can be normalized when the sensors 210, 212 are in a pre-determined orientation with respect to one another. For example, in the pre-determined orientation, the sensors 210, 212 is spaced from one another by a known distance, the sensor 210 is longitudinally aligned with the pressure-sensing location 208, etc.

The intravascular instrument 202 may be advanced through a lumen of the intravascular instrument 204 en route to a stenosis or other intravascular location, and alignment of the intravascular instruments 202 and 204 may occur at one or more points during the advancement. For example, the intravascular instruments 202 and 204 may be spatially aligned when the sensor 210 is longitudinally aligned with the pressure sensing location 208, as shown, for example, in FIG. 6. Though the pressure sensing location 208 is depicted as being located at an ostium at a distal end of the intravascular instrument 204, the pressure sensing location 208 may be located elsewhere on the intravascular instrument 204, e.g., some distance proximal of the distal end of intravascular instrument 204. Alignment may be satisfied despite some margin of error, e.g., within 1 cm, 2 cm, 3 cm, 4 cm, or 5 cm of an alignment described herein. For example, the intravascular instruments 202 and 204 may be aligned when the sensor 210 is within 3 cm of the pressure sensing location 208. For the sake of brevity, and without limiting the scope of the disclosure, alignment will generally be discussed herein without reference to margins of error.

In some embodiments, the pressure sensors 210 and 212 are part of a single intravascular device. Exemplary embodiments are described, example, in U.S. Pat. No. 6,106,476 and U.S. Publication No. 2014/0180032, the entireties of which are hereby incorporated by reference herein. While some embodiments of the present disclosure refer to multiple intravascular instruments, in some embodiments, only one intravascular instrument is utilized. In some embodiments, the pressure sensors 210, 212 may be movable relative to one another. Exemplary embodiments are described in U.S. Publication No. 2013/0345574, the entirety of which is hereby incorporated by reference herein. In some embodiments, both pressure sensors 210, 212 can be positioned within the body 220 of the patient. In some embodiments, the pressure sensors 210, 212 are movable relative to one another while the sensors 210, 212 are positioned within the body 220. Normalization can be automatically initiated when the pressure sensors 210, 212 are in a pre-determined orientation relative to one another, such as when the pressure sensors 210, 212 are longitudinally aligned with respect to one another.

A medical processing unit, which may in some instances comprise the computing device 172, may be in communication with one or both of the pressure sensors 210, 212 and may detect when the pressure sensors 210, 212 are in a pre-determined configuration relative to one another and automatically initiate normalization of the sensor 210 and the sensor 212 of the intravascular instrument 204 in response. In an embodiment, the medical processing unit may be in communication with a radiographic imaging source such as the radiographic unit 186 and may receive radiographic data, e.g., angiographic and/or fluoroscopic images, from the radiographic imaging source. In some cases, the medical processing unit may detect when the intravascular instruments 202 and 204 are aligned based on radiographic images and/or other radiographic data received from the radiographic imaging source. For example, the medical processing unit can be configured to identify the location of the sensor 210 and/or the pressure sensing location 208 in the radiographic images and to determine when the sensor 210 and the pressure sensing location 208 are longitudinally aligned.

For example, the radiopaque region 214 of intravascular instrument 202 may present a signature radiographic profile when the intravascular instruments 202 and 204 are aligned, and the medical processing unit may analyze the radiographic images in search of said radiographic profile. In an embodiment, the radiopaque region 214 may be of a certain length, e.g., 1 cm, 2 cm, 3 cm, 4 cm, 5 cm, or longer than 5 cm, and may extend from a distal tip of the intravascular instrument 202 at its distal end to the sensor 210 at its proximal end. Being that the sensor 210 is located at the proximal end of the radiopaque region 214, the medical processing unit may determine that the intravascular instruments 202 and 204 are aligned when the full length, or some other portion that would result in alignment within the margin of error, of the radiopaque region 214 protrudes from the distal end of the intravascular instrument 204. Accordingly, in some cases, detecting and/or determining that the intravascular instruments 202 and 204 are aligned may comprise detecting a radiographic pattern or profile in a radiographic image that matches a radiographic pattern or profile associated with the intravascular instruments 202 and 204 in an aligned state. The medical processing unit may maintain in its memory or in a remote database an archive of such patterns or profiles.

Although only intravascular instrument 202 is shown as comprising the radiopaque region 214, it is understood that intravascular instrument 204 may likewise comprise one or more radiopaque regions. It is further understood that while the radiopaque region 214 is only shown as being located distal of the sensor 210, one or more radiopaque regions 214 may be located proximal of the sensor 210 as well. As shown in FIG. 7, radiopaque regions 214 may be separated by non-radiopaque regions 216. In that regard, alternating radiopaque regions 214 and non-radiopaque regions 216 may be the same length or may be different lengths. In some cases, different radiopaque patterning may exist on different sides of the sensor 210. The radiopaque region 214 may allow the medical processing unit to determine the location of the sensor 210. In some cases, the medical processing unit may determine whether or not the intravascular instruments 202 and 204 are aligned based on the location of the sensor 210. For example, the medical processing unit may determine whether or not intravascular instruments 202 and 204 are aligned based on whether the sensor 210 is aligned with the pressure sensing location 208.

In an embodiment, the medical processing unit may detect when the sensors 210, 212 are approaching the pre-determined orientation and may prompt an operator controlling the progression of the intravascular instrument 202 and/or intravascular instrument 204 through the vasculature of a patient. The prompt may be visual, audible, alphanumeric, textual, tactile, or any other suitable prompt capable of notifying the operator that the intravascular instruments 202 and 204 are nearing alignment. Once the operator is aware that the intravascular instruments 202 and 204 are nearly aligned, the operator can moderate the speed, angle, orientation, or other aspect of progression through the vasculature to reduce the likelihood that alignment will be overshot, bypassed, or otherwise passed over before normalization is completed. The medical processing unit may be able to perceive the radiopaque region 214 of the intravascular instrument 202 through the outer surfaces of intravascular instrument 204 as the intravascular instrument 202 moves through a lumen of the intravascular instrument 204, which may facilitate the medical processing unit detecting when the intravascular instruments 202 and 204 are approaching alignment. In an embodiment, the medical processing unit may track the intravascular instruments 202 and 204 as one or the other or both moves through the vasculature of a patient. Such tracking may facilitate one or the other or both of prompting an operator when the intravascular instruments 202 and 204 are nearing alignment and detecting that the intravascular instruments 202 and 204 are aligned.

As discussed above, the medical processing unit may automatically normalize the sensor 210 and the sensor 212 of the intravascular instrument 204 to each other once the medical processing unit detects that the sensors 210, 212 are in the pre-determined orientation. Automatic normalization may be performed without instructions from the operator. Accordingly, automatic normalization beneficially reduces the risk that the operator will forget to perform the normalization step prior to assessing a vessel and thereby reduces the risk that data obtained during vessel assessment, e.g., pressure measurements, will contain errors resulting from failure to perform the normalization. Automatically initiating normalization also advantageously improves the efficiency of the workflow by eliminating a manual step of initiating normalization that would have to be performed by a medical professional. In some embodiments, the medical processing unit may present an operator with an option to activate or deactivate the automatic normalization functionality.

Normalization may occur instantaneously or over some period of time, e.g., half a second, 1 second, 2 seconds, 3 seconds, 4 seconds, 5 seconds, 10 seconds, 30 seconds, 1 minute, 1 cardiac cycle, 2 cardiac cycles, 3 cardiac cycles, 4 cardiac cycles, 5 cardiac cycles, 6 cardiac cycles, 7 cardiac cycles, 8 cardiac cycles, 9 cardiac cycles, 10 cardiac cycles, or over some other period of time. Normalization can be performed using any suitable number of heart cycles preceding or follow/occurring after initiation of the normalization. The medical processing unit may prompt the operator not to move or otherwise disturb the intravascular instruments 202 and 204 during the normalization process as moving the either of the intravascular instruments 202 and 204 during normalization could destroy the integrity of the normalization and lead to errors in data collection. The prompt may be visual, audible, alphanumeric, textual, tactile, or any other suitable prompt capable of notifying the operator that normalization is taking place. Similarly, the medical processing unit may prompt the operator when normalization has completed. The prompt may be visual, audible, alphanumeric, textual, tactile, or any other suitable prompt capable of notifying the operator that normalization has completed.

After assessment of one or more areas of interest, e.g., a stenosis, within a vessel, the medical processing unit may calculate a drift that has occurred between the sensor 210 and a sensor of the intravascular instrument 204. The drift may be calculated whether or not normalization was performed prior to gathering data to assess the vessel. For example, the medical processing unit may record that pressure measurements from the sensor 210 and the sensor of the intravascular instrument 204 are not calibrated to each other and may note the character of the disparity prior to assessment of the vessel and may record a change in the character of the disparity after assessment. The medical processing unit may calculate the drift that occurred between the pre-assessment and post-assessment records, and may adjust the gathered data accordingly. Similarly, when the sensor 210 and the sensor of the intravascular instrument 204 were normalized prior to assessment of the vessel, the medical processing unit may record that a drift has occurred between the pre-assessment normalization and post-assessment recording of the change. Since the sensor 210 and the sensor of the intravascular instrument 204 were normalized prior to assessment of the vessel, the medical processing unit may calculate the drift by calculating the post-assessment disparity. As discussed above, the medical processing unit may adjust the gathered data to account for the disparity.

Turning now to FIGS. 8A and 8B, shown therein are a plurality of fluoroscopic images, including image 300 and image 302. As described hereinabove, the medical processing unit may receive radiographic images from a radiographic imaging source, e.g., the radiographic unit 186. The medical processing unit may output such radiographic images, e.g., images 300 and 302, to a display such as the display device 180. Image 300 comprises an unenhanced image of the intravascular instruments 202 and 204. In some cases, as shown in image 302, the medical processing unit may enhance depiction of one or the other or both of the intravascular instruments 202 or 204 in displayed images. For example, the medical processing unit may indicate with circle 222 that the intravascular instrument 202 and intravascular instrument 204 are aligned or are nearing alignment.

In an embodiment, the medical processing unit enhances radiopaque regions, e.g., radiopaque region 214, of the intravascular instruments 202 and 204. In an embodiment, the medical processing unit may enhance depiction of intravascular instruments 202 and 204 by highlighting the intravascular instrument 202 with a first color and highlighting the intravascular instrument 204 with a second color. Though not shown, the medical processing unit may display one or more prompts discussed hereinabove. For example, the medical processing unit may display a prompt indicating that the intravascular instruments 202 and 204 are nearing alignment, that normalization is underway, that normalization has completed, or combinations thereof. In some cases, the prompts may include highlighting or changing the highlighting of depiction of the intravascular instruments 202 and 204. For example, one or the other or both of the intravascular instruments 202 and 204 may be highlighted in red while normalization is underway and such highlighting may change to green when normalization is completed.

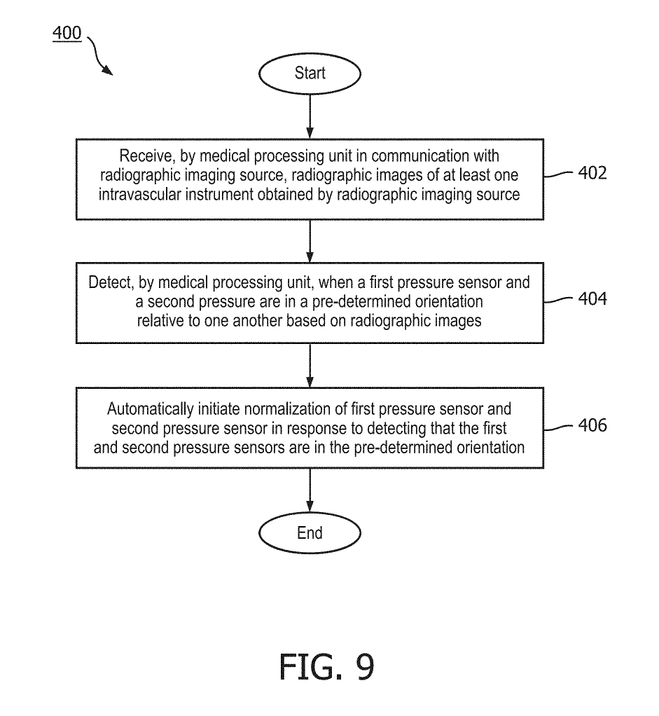

Referring now to FIG. 9, shown therein is a flow chart of a method 400 according to embodiments of the disclosure. Portions of the method 400 may correspond to techniques discussed hereinabove with reference to FIGS. 1-8B and may be performed with hardware and/or software components of the system 150, the intravascular instrument 202, the intravascular instrument 204, or combinations thereof. The method 400 begins at block 402 where radiographic images of at least one intravascular instrument obtained by the radiographic imaging source are received by a medical processing unit in communication with a radiographic imaging source. In some embodiments, the at least one intravascular instrument is a single instrument comprises two or more pressure sensors. In other embodiments, the at least one intravascular instrument comprises a first intravascular instrument and a second intravascular instrument, each having their own associated pressure sensor. In some instances, the medical processing unit may comprise the computing device 172, the first and second intravascular instruments may comprise the intravascular instruments 202 and 204, respectively, the radiographic imaging source may comprise the radiographic unit 186, or combinations thereof. The method 400 may in some cases include displaying the radiographic images and visually enhancing depiction of the first and second intravascular instruments in the displayed radiographic images. In some cases, visually enhancing depiction of the first and second intravascular instruments may comprise highlighting the first intravascular instrument with a first color and highlighting the second intravascular instrument with a second color.

The method 400 continues at block 404 where the medical processing unit detects when the first pressure sensor is in a pre-determined orientation relative to the second pressure sensor based on the radiographic images. For example, the first pressure sensor may be longitudinally aligned with the second pressure sensor. In some embodiments, the first pressure sensor may be longitudinally aligned with a pressure-sensing location in communication with the second pressure sensor. In some cases, detecting when the first and second intravascular instruments are aligned may be based on a radiopaque region of at least one of the first or second intravascular instruments. In some cases, detecting when the first and second intravascular instruments are aligned may comprise determining when the first pressure sensor is aligned with a pressure sensing location of the second intravascular instrument. In some cases, detecting when the first and second intravascular instruments are aligned may comprise determining when the first pressure sensor is aligned with an ostium of the second intravascular instrument. In some implementations, the method 400 may include prompting an operator when the first and second intravascular instruments are near alignment. Prompting the operator when the first and second intravascular instruments are near alignment may allow the operator to moderate the pace at which the first and/or second intravascular instrument is being moved through a vessel and thereby reduce the chance that an alignment configuration will be overshot, bypassed, or otherwise passed over. The method 400 may further include tracking, by the medical processing unit, the locations of the first and second intravascular instruments within a body lumen while at least one of the first or second intravascular instruments is moved through the body lumen.

The method 400 continues at block 406 where normalization of a first pressure sensor and a second pressure sensor is automatically initiated in response to detecting that the first and second pressure sensors are in the pre-determined orientation. In some implementations, the method 400 may include prompting an operator not to disturb at least one intravascular instrument during the normalization process. Such prompting may reduce the likelihood of errors in normalization resulting from movement of one or both of the first and second intravascular instruments. Similarly, in some implementations, the method 400 may include prompting an operator when normalization is completed. Though not shown in FIG. 9, the method may further comprise additional steps consistent with the foregoing disclosure. Further, the method may omit some of the steps shown in FIG. 9 and/or perform the steps in various orders without departing from the scope of the present disclosure.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A medical system, comprising:
a medical processing unit in communication with a first pressure sensor, a second pressure sensor, and a radiographic imaging source configured to obtain radiographic images of at least one intravascular instrument positioned within a body lumen, wherein the medical processing unit is configured to:
receive the radiographic images obtained by the radiographic imaging source;
detect, using the radiographic images, when the first pressure sensor is in a pre-determined orientation with respect to the second pressure sensor; and
automatically initiate normalization of the first pressure sensor and the second pressure sensor in response to detecting that the first pressure sensor is in the pre-determined orientation with respect to the second pressure sensor.

2. The medical system of claim 1, further comprising:
a first intravascular instrument, wherein the first pressure sensor is configured to measure a pressure at a distal portion of the first intravascular instrument.

3. The medical system of claim 2, wherein the first pressure sensor is disposed at the distal portion of the first intravascular instrument.

4. The medical system of claim 3, further comprising:
a second intravascular instrument, wherein the second pressure sensor is configured to measure a pressure at a distal portion of the second intravascular instrument.

5. The medical system of claim 4, wherein the second pressure sensor is disposed at a proximal portion of the second intravascular instrument and in communication with a pressure-sensing location at a distal portion of the second intravascular instrument.

6. The medical system of claim 5, wherein the medical processing unit is configured to detect when the first and second pressure sensors are in the pre-determined orientation by determining when the first pressure sensor is aligned with the pressure sensing location of the second intravascular instrument.

7. The medical system of claim 5, wherein pressure sensing location of the second intravascular instrument comprises an ostium at the distal portion.

8. The medical system of claim 7, wherein the medical processing unit is configured to detect when the first and second pressure sensors are in the pre-determined orientation by determining when the first pressure sensor is aligned with the ostium of the second intravascular instrument.

9. The medical system of claim 7, wherein the second intravascular instrument comprises a catheter.

10. The medical system of claim 3, wherein the first intravascular instrument comprises a guide wire.

11. The medical system of claim 4, wherein the medical processing unit is configured to detect when the first and second pressure sensors are in the pre-determined orientation based on a radiopaque region of at least one of the first or second intravascular instruments.

12. The medical system of claim 4, wherein the medical processing unit is further configured to track locations of the first intravascular instrument and second intravascular instrument within the body lumen while at least one of the first or second intravascular instruments is being moved through the body lumen.

13. The medical system of claim 1, wherein the medical processing unit is further configured to prompt an operator when the first and second sensors are nearing the pre-determined orientation.

14. The medical system of claim 4, wherein the medical processing unit is further configured to prompt an operator to refrain from moving the first and second intravascular instruments in response to detecting that the first pressure sensor is in the pre-determined orientation with respect to the second pressure sensor.

15. The medical system of claim 4, wherein the medical processing unit is further configured to display the radiographic images and to visually enhance depiction of the first and second intravascular instruments in the displayed radiographic images.

16. The medical system of claim 4, wherein the medical processing unit is configured to visually enhance depiction of the first and second intravascular instruments by highlighting the first intravascular instrument with a first color and highlighting the second intravascular instrument with a second color.

17. A method, comprising:
receiving, by a medical processing unit in communication with a radiographic imaging source, radiographic images of at least one intravascular instrument obtained by the radiographic imaging source;
detecting, by the medical processing unit, when a first pressure sensor is in a pre-determined orientation with respect to a second pressure sensor based on the radiographic images; and
automatically initiating normalization of the first pressure sensor and the second pressure sensor in response to detecting that the first pressure sensor is in the pre-determined orientation with respect to the second pressure sensor.

18. The method of claim 17, wherein the at least one intravascular instrument comprises a first intravascular instrument and a second intravascular instrument, the first pressure sensor associated with the first intravascular instrument and the second pressure sensor associated with the second intravascular instrument, and wherein the detecting comprises:
determining when the first pressure sensor is aligned with a pressure sensing location of the second intravascular instrument, the second pressure sensor in communication with the pressure sensing location.

19. The method of claim 18, further comprising:
outputting, by the medical processing unit, data representative of a prompt to prompt an operator to refrain from moving the first and second intravascular instruments in response to detecting that the first pressure sensor is in the pre-determined orientation with respect to the second pressure sensor.

20. The method of claim 18, further comprising displaying the radiographic images and visually enhancing depiction of the first and second intravascular instruments in the displayed radiographic images.
